(19) United States
(12) Patent Application Publication
(10) Pub. No.: US 2007/0167877 A1
(43) Pub. Date: Jul. 19, 2007

(54) Medical catheters and methods

(76) Inventors: Charles Louis Euteneuer, St. Michael, MN (US); Duane G. Frion, Brooklyn Center, MN (US)

Correspondence Address: CYR & ASSOCIATES, P.A., PONDVIEW PLAZA, 5850 OPUS PARKWAY SUITE 114, MINNETONKA, MN, 55343, US

(21) Appl. No.: 11/333,045

(22) Filed: Jan. 17, 2006

(51) Int. Cl. A61M 25/00

(52) U.S. Cl. 600/585

(57) ABSTRACT

Catheters for introducing fluids into a bodily lumen of a patient are disclosed. The fluids may include medicinal, imaging or other compounds. The catheters include a proximal tube, a core wire and a distal tube. The first end of the core wire is secured to the second end of the proximal tube. The core wire extends through the distal tube. A proximal lumen of the proximal tube is in communication with the distal lumen of the distal tube. A bridge tube may be provided between the proximal lumen and the distal lumen to facilitate communication of the proximal lumen and the distal lumen. The catheter may be configured with a relatively small diameter and low overall profile using aspects of the present inventions. The catheters may include a balloon at a distal end to receive the fluid. The fluid provided through the catheter may inflate the balloon. The catheter may include a distal outlet release a fluid into the patient.

### BACKGROUND OF THE INVENTION

1. Summary of the Invention

The present inventions relate to medical devices and, more particularly, to medical catheters and medical guidewires for insertion into bodily lumen of patients.

2. Description of the Related Art

Medical catheters and guidewires can be useful tools in treating intravascular disorders, disorders within other lumen of the body, extracting fluids from lumen as well as introducing fluid into lumen. Some medical catheters and most guidewires are configured to be received through a medical device to permit the medical device to be slid over the medical catheter or guidewire and positioned within the body of a patient. Further, many catheters and some guidewire designs include a balloon at or near the distal end of the catheter or guidewire. Depending on the configuration, these devices can also be used to introduce and/or expand various other medical devices, such as stents for example. The balloons may help direct the distal end of the catheter through a lumen where the pulsatile flow of blood may permit them to act as a “sail.” Further, the balloons in various configurations may be used to test for the occlusion of vessels, for embolization for bleeding, to treat or control vasospasms, and for treatment of nosebleeds, among other uses.

Medical catheters and guidewires are particularly useful in accessing remote and tortuous locations within the body. Because of the need to navigate through the body to remote locations through narrow twisting lumen, medical catheters and guidewires are frequently long thin devices. The materials and configurations of the tubular devices frequently vary along their length to provide adequate torquability and pushability to guide the distal portions of the device within the body. These variations can result in a number of transitions along the length of the medical catheters and guidewires. These transitions frequently involve the junction of two or more tubes. At these tubular junctions, the central lumen for communication of fluids must permit the flow of fluids and typically needs to maintain the desired torquability and pushability for the device as a whole. Accordingly, a need exists for junctions which permit the communication of fluids while maintaining the desired performance characteristics.

Further, there are significant benefits in reducing the diameter of medical catheters and guidewires for many applications. The reduced size is generally less traumatic to a patient. The reduced size also permits access to locations of reduced size or diameter that may not be reachable or treatable by larger diameter medical catheters and guidewires. Various locations in the brain or heart can be particularly difficult to reach and/or treat when the area to be treated or otherwise accessed is in the. This is, at least in part, due to the tortuous path that must be navigated to access some locations as well as the point for introduction of the medical catheter or guidewire frequently being the femoral artery. Reduced diameter medical catheters and guidewires may have physical characteristics that may enhance the difficulty of placing them at such remote locations. Accordingly, a need exists for interconnections that provide performance characteristics that simplify the placement of reduced diameter medical catheters and guidewires.

Manufacturing medical devices can be difficult. As the size of the devices is decreased the difficulty in manufacturing the devices generally goes up. Medical catheters and guidewires typically have outside diameters as small as about 0.0010 inches. Accordingly, a need exists for interconnections that provide for simplified manufacture of reduced diameter medical catheters and guidewires.

### SUMMARY OF THE INVENTION

Apparatus and methods in accordance with the present invention may resolve many of the needs and shortcomings discussed above and provide additional improvements and advantages as will be recognized by those skilled in the art upon review of the present disclosure.

The present invention provides a catheter in the form of two or more elongated hollow tubes secured to one another. A typical catheter in accordance with the present invention includes a proximal tube connected to a distal tube. The proximal tube defining a proximal lumen and the distal tube defining a distal lumen. The proximal lumen and the distal lumen are in fluid communication with one another. A core wire is provided and positioned through at least a portion of the distal tube. In one aspect, the core wire may be provided and positioned through the distal lumen. In some aspects, the catheter may have a balloon secured to its distal end. The balloon in communication with at least one lumen for purposes of inflation.

In one aspect, small bodily lumens of a patient can be accessed with a catheter in accordance with the present invention, by conventional guidewire techniques. Small bodily lumens of the body can be accessed by the catheter to provide certain types of medical diagnosis and/or treatment at the desired location within the body. In some cases, the catheter may have a diameter to enable conventional over-the-wire instruments to be passed over the catheter.

The tubes may be formed by extrusion and drawing and typically have a sufficiently stiff proximal end, a flexible, atraumatic distal end, and a wall thickness to optimize the cross-sectional area of the lumen for particular applications. In one aspect, the proximal tube and/or the distal tube can be formed from stainless steel. In another aspect, the proximal tube and/or the distal tube can be formed from nitinol. One or both of the proximal tube and the distal tube may be annealed progressively to vary the flexibility along the length of the proximal and the distal tubes. The distal portions of the proximal tube may be annealed such that the distal portions has greater flexible than proximal portions of the distal tube.

In one aspect, an apparatus in accordance with the present invention may be a medical catheter or medical guidewire including a proximal tube, a core wire and a distal tube. The proximal tube may define a proximal lumen extending between a first end and a second end of the proximal tube. The second end of the proximal tube may include a notch defined by one or more notch surfaces. The distal tube defines a distal lumen and may also define a core wire lumen. The core wire includes a first end and a second end. The core wire may also include a core wire mating surface extending to the first end of the core wire. The core wire mating surface may be secured within the notch of the proximal tube. The core wire may be received within the distal lumen or the core wire lumen of the distal tube. The distal lumen of the distal tube is in fluid communication with the proximal lumen of the proximal tube. A bridge tube may be included to permit or enhance the communication of fluid between the proximal lumen and the distal lumen. The bridge tube defining a bridge lumen.

In one aspect, the notch surfaces defining the notch may include a longitudinal notch surface and the core wire mating surface may include a longitudinal mating surface. In this aspect, the longitudinal notch surface may be secured to the longitudinal mating surface over at least a portion of the length of the longitudinal notch surface. In another aspect, the notch surface defining the notch may include a perpendicular notch surface and the core wire mating surface may include a perpendicular mating surface. In this aspect, the perpendicular notch surface may be secured to the perpendicular mating surface over at least a portion of the length of the perpendicular notch surface. In another aspect, the notch surface defining the notch may include both a longitudinal notch surface and a perpendicular notch surface and the core wire mating surface may include a longitudinal mating surface and a perpendicular mating surface. In this aspect, the longitudinal notch surface may be secured to the longitudinal mating surface over at least a portion of the length of the longitudinal notch surface and the perpendicular notch surface may be secured to the perpendicular mating surface over at least a portion of the length of the perpendicular notch surface. In another aspect, the notch surface defining the notch may include an angled notch surface and the core wire mating surface includes an angled mating surface. In this aspect, the angled notch surface may be secured to the angled mating surface over at least a portion of the length of the angled notch surface. In addition, the core wire may include a flow facilitating surface.

An atraumatic tip secured to the distal end of the distal tube may have a hemi-spherical or rounded tip for atraumatic insertion into the body. The atraumatic tip may be fabricated from a metal or may be a polymeric material such as PET, polyimide, or polyethylene. The atraumatic tip may include a shaping wire secured to or within the distal end of the distal tube. A coil may extend around the shaping wire. The rounded tip may be secured to one or both of the coil and the shaping wire. The atraumatic tip may include one more components containing a radio-opaque material.

In another aspect, the present inventions may provide methods of treating a patient using the catheter. The catheter is inserted into a bodily lumen and guided to a target location in the lumen requiring treatment. Once positioned at the target location, a fluid is introduced into the patent, a fluid is removed from the patient and/or a balloon may be inflated. In one aspect, inflation media is passed through the catheter and exits the catheter though one or more of the plurality of openings in the distal tube of the catheter to inflate the balloon. In other aspects, therapeutic or diagnostic agents may be passed through the catheter and exit the catheter though one or more of the plurality of openings in the distal tube of the catheter to enter the bodily lumen of a patient at the desired location. Further, a surgical instrument may be slid, in guided contact, over the catheter to access a desired location during a surgical operation. After the treatment, the distal balloon, if present, is typically deflated by withdrawing inflation media through one or more of the plurality of slots. The catheter is removed from the body lumen.

Other features and advantages of the invention will become apparent from the following detailed description, and from the claims.

### BRIEF DESCRIPTION OF THE DRAWINGS

FIG. 1 illustrates a partial perspective view of an exemplary embodiment of a catheter in accordance with the present inventions;

FIG. 2 illustrates a partial side view in cross-section of an exemplary embodiment of an interconnection between a proximal tube and a distal tube in accordance with the present inventions;

FIG. 3 illustrates a partial side view in cross-section of another exemplary embodiment of an interconnection between a proximal tube and a distal tube in accordance with the present inventions;

FIG. 4 illustrates a partial perspective view of an exemplary embodiment of a notch in the proximal tube in accordance with the present inventions similar to those illustrated in FIGS. 1, 2, 3, 7 and 9;

FIG. 5 illustrates a partial perspective view of an exemplary embodiment of a core wire mating surface of a core wire in accordance with the present inventions similar to those illustrated in FIGS. 2 and 9;

FIG. 6A illustrates a transverse cross-section through section lines 6A-6A of the embodiment of the interconnection illustrated in FIG. 2;

FIG. 6B illustrates a transverse cross-section through section lines 6B-6B of the embodiment of the interconnection illustrated in FIG. 2;

FIG. 6C illustrates a transverse cross-section through section lines 6C-6C of the embodiment of the interconnection illustrated in FIG. 2;

FIG. 7 illustrates a partial side view in cross-section of another exemplary embodiment of an interconnection between a proximal tube and a distal tube in accordance with the present inventions;

FIG. 8A illustrates a transverse cross-section through section lines 8A-8A of the embodiment of the interconnection illustrated in FIG. 7;

FIG. 8B illustrates a transverse cross-section for an exemplary alternative lumen configuration through an interconnection similar to embodiment of FIG. 7;

FIG. 9 illustrates a partial side view in cross-section of another exemplary embodiment of an interconnection between a proximal tube and a distal tube in accordance with the present inventions;

FIG. 10A illustrates a transverse cross-section through section lines 10A-10A of the embodiment of the interconnection illustrated in FIG. 9;

FIG. 10B illustrates a transverse cross-section through section lines 10B-10B of the embodiment of the interconnection illustrated in FIG. 9;

FIG. 11 illustrates a partial side view in cross-section of another exemplary embodiment of an interconnection between a proximal tube and a distal tube in accordance with the present inventions showing;

FIG. 12 illustrates a partial perspective view of an exemplary embodiment of a notch in the proximal tube in accordance with the present inventions similar to those illustrated in FIG. 11;

FIG. 13 illustrates a partial perspective view of an exemplary embodiment of a core wire mating surface of a core wire in accordance with the present inventions similar to those illustrated in FIG. 11;

FIG. 14A illustrates a cross-section through shown by section lines 10A-10A of the embodiment of the interconnection illustrated in FIG. 11;

FIG. 14B illustrates a cross-section through shown by section lines 10B-10B of the embodiment of the interconnection illustrated in FIG. 11;

FIG. 15A illustrates a cross-section through embodiments of a balloon, a distal tube and a guidewire at the distal end of a catheter in accordance with the present invention;

FIG. 15B illustrates a cross-section through other embodiments of a balloon, a distal tube and a guidewire at the distal end of a catheter in accordance with the present invention; and

FIG. 15C illustrates a cross-section through other embodiments of a distal tube and a guidewire at the distal end of a catheter in accordance with the present invention.

All Figures are illustrated for ease of explanation of the basic teachings of the present invention only; the extensions of the Figures with respect to number, position, relationship and dimensions of the parts to form the preferred embodiment will be explained or will be within the skill of the art after the following description has been read and understood. Further, the exact dimensions and dimensional proportions to conform to specific force, weight, strength, and similar requirements will likewise be within the skill of the art after the following description has been read and understood.

Where used in various Figures of the drawings, the same numerals designate the same or similar parts. Furthermore, when the terms “top,” “bottom,” “right,” “left,” “forward,” “rear,” “first,” “second,” “inside,” “outside,” and similar terms are used, the terms should be understood to reference only the structure shown in the drawings and utilized only to facilitate describing the illustrated embodiments. Similarly, when the terms “proximal,” “distal,” and similar positional terms are used, the terms should be understood to reference the structures shown in the drawings as they will typically be utilized by a physician or other user who is treating or examining a patient with an apparatus in accordance with the present invention.

### DETAILED DESCRIPTION OF THE INVENTION

The figures generally illustrate exemplary embodiments of catheters 10 or components thereof which include aspects of the present inventions. The particular embodiments of the catheters 10 illustrated in the figures have been chosen for ease of explanation and understanding of various aspects of the present inventions. These illustrated embodiments are not meant to limit the scope of coverage but instead to assist in understanding the context of the language used in this specification and the appended claims. Accordingly, many variations from the illustrated embodiments may be encompassed by the appended claims.

The present inventions provide catheters 10 and methods for directing fluids to a target location within a patient and, in some cases, withdrawing bodily fluids from a target location within a patient. In certain aspects, catheters 10 in accordance with the present inventions may permit the passage of fluid through a lumen while maintaining a relatively small outside diameter. The catheter 10 may introduce the fluid locally or systemically into the patient. These fluids may include medicinal compounds, such as electrolytes and pharmaceuticals; imaging compounds; cells; compositions for gene transfer; or other compounds or compositions benefiting from localized or systemic introduction through a catheter 10. The catheter 10 may alternatively introduce the fluid into a balloon 20. Fluids introduced into balloons 20 may be various types of inflation media which will be recognized by those skilled in the art upon review of the present disclosure. Inflation media may include various imaging compounds, medicinal compounds, or other compounds depending upon the function and configurations of the balloon 20. The fluid may be used solely to expand a balloon 20 secured to the catheter 10. The fluid may be directed through the catheter 10 into an inflation chamber 30 defined by a balloon 20. The catheter 10 may also be used to withdraw fluid from the inflation chamber 30 to permit the deflation of a balloon 20.

As generally illustrated throughout the Figures, catheters 10 generally include a proximal tube 12, a core wire 14 and a distal tube 16. Catheters 10 may also include a bridge tube 18. The proximal tube 12 and distal tube 16 are generally secured together coaxially. The proximal tube 12, distal tube 16, and, if present, bridge tube 18 are configured such that their lumen define a passage for communication of fluids extending from the proximal end to the distal end of catheters 10. The core wire 14 extends through the distal tube and is secured at a first end to the proximal tube. An atraumatic tip 90 may be provided at the second end 214 of the core wire 14 or at the second end 216 of the distal tube 16. Catheters 10 generally define a longitudinal axis 300 extending along their length.

The proximal tube 12 is generally configured to guide and position portions of the distal tube 16 within a patient. The proximal tube 12 cooperates with the core wire 14 to position the distal tube 16. The proximal tube 12 defines a proximal lumen 22 extending over at least a portion of the length of the proximal tube 12. The proximal lumen 22 of the proximal tube 12 generally extends longitudinally within the proximal tube 12 from a first proximal tube opening 32 to a second proximal tube opening 42 defined by the proximal tube 12. The proximal lumen 22 may extend between a first proximal tube opening 32 and a second proximal tube opening 42 defined by the proximal tube 12. The proximal lumen 22 of the proximal tube 12 is generally configured to receive a fluid, such as for example inflation media, and communicate the fluid at least toward a second end 212 of a proximal tube 12.

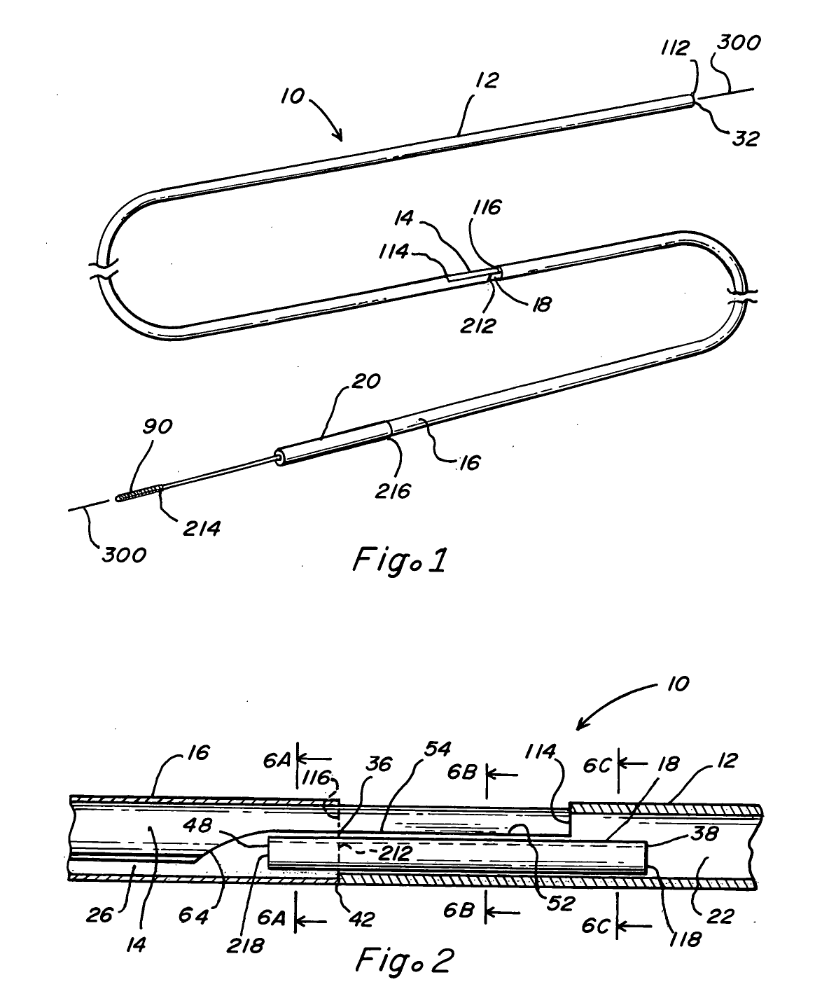

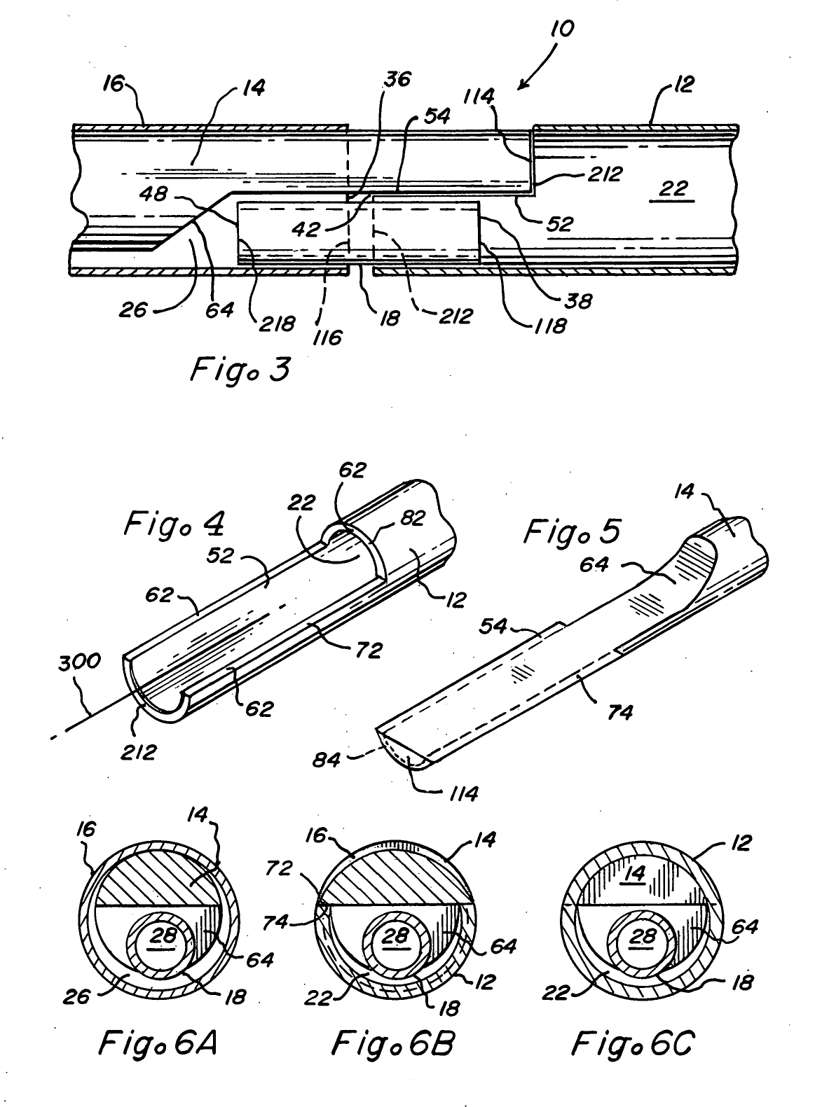

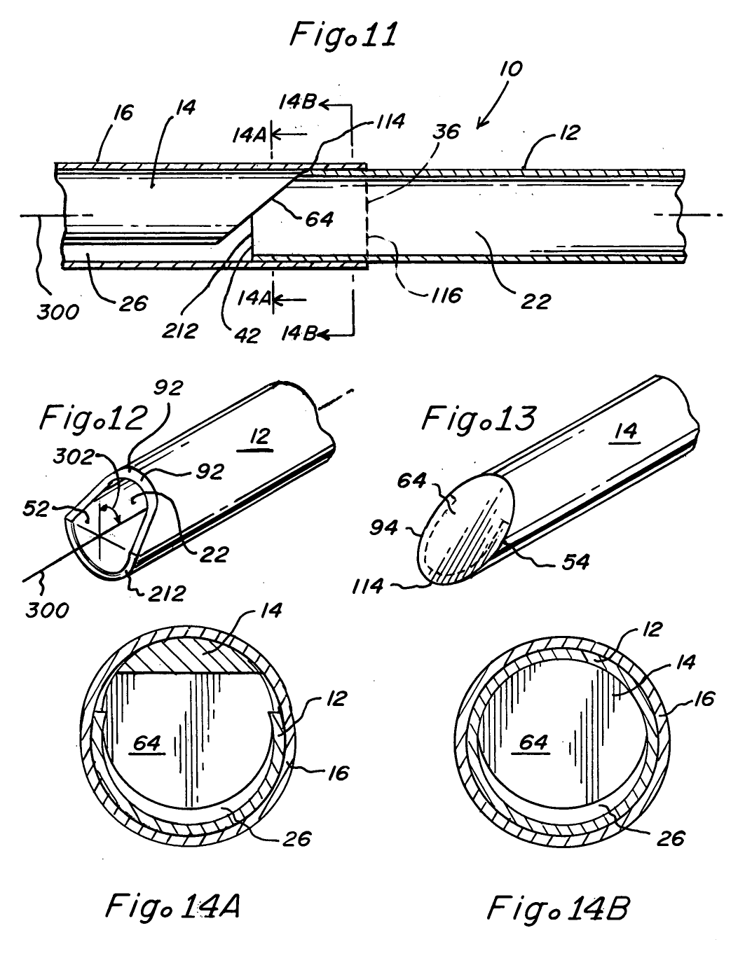

The proximal tube 12 includes one or more notch surfaces 62 of proximal tube 12 defining the notch 52 at a second end 212 of the proximal tube 12. The first end 214 of the core wire 14 is secured in the notch 52 at the second end 212 of the proximal tube 12. The notch 52 may extend into the proximal lumen 22 as can be readily discerned from FIGS. 4 and 12. The notch 52 is generally configured to permit fluid to flow through the proximal lumen 22 to or from the second proximal tube opening 42 of the proximal tube 12 when the first end 114 of a guidewire is secured within the notch 52. The notch 52 generally extends from the second end 212 of the proximal tube 12 to a location along the tube which is proximal to the second end 212 of the proximal tube 12. The notch 52 is generally shaped to receive a core wire mating surface 54, outlined in and readily discernable from FIGS. 5 and 13, at a first end 214 of core wire 14. The notch surfaces 62 may be shaped to correspond to the shape of the core wire mating surface 54 or vice versa. The notch surfaces 62 are frequently configured to optimize their interaction with the core wire mating surface 54 to provide the desired performance characteristics to the resultant catheter 10. In some aspects, the notch 52 may extend to or through the longitudinal axis 300 of the proximal tube 12. In another aspect, the notch 52 does not extend through the proximal tube to the longitudinal axis 300 of the proximal tube 12. The notch surface 62 may define a plane and may define two or more planes. As illustrated, in FIGS. 1 to 10, 14A and 14B, the notch 52 is defined by a plurality of notch surfaces 62. The notch surfaces 62 of FIGS. 1 to 10, 14A and 14B generally include a longitudinal notch surface 72 cooperating with a perpendicular notch surface 82 to form the notch 52. The notch 52 may also be defined by a single planar notch surface 62. For example, notch surface 62 may be at an angle 302 relative to the longitudinal axis 300 to form an angled notch surface 92 as is generally illustrated in FIGS. 11 and 12.

The proximal tube 12 may be made from a variety of materials including polymers, metals, and various composite materials. In one aspect, the proximal tube 12 is made of a stainless steel. In another aspect, the proximal tube 12 is made of nitinol. The proximal tube 12 may be configured to have a desired balance of longitudinal stiffness and torsional rigidity based on the combined characteristics of the distal tube 16 and core wire 14. The longitudinal stiffness at least in part dictates the push characteristics for the proximal tube 12. The torsional rigidity at least in part dictates the precision of the rotational control provided by the proximal tube 12. Typically, the proximal tube is configured to have a desired elastic range.

The proximal tube 12 may have various diameters and lengths depending on the particular application for the catheter 10. When catheter 10 includes a balloon 20, the proximal tube 12 is generally configured to support inflation of the particular type of balloon 20. The particular configuration of proximal tube 12 may also depend upon whether or not the proximal tube 12 is intended to use this device as the primary balloon catheter or as a wire support for other catheters. For use primarily as a balloon catheter, a catheter 10 is typically configured to support larger volumes of fluid than when the catheter 10 used as a delivery rail for other devices. In such applications for balloon inflation, the tube may have an outside diameter of about 0.024 inches and a lumen diameter of about 0.019 inches. This outside diameter can provide the desired torsional rigidity without being too longitudinally stiff. The diameter of the proximal lumen 22 may be selected to provide a desire inflation/deflation time. For intercranial applications where the insertion point is in the femoral artery, the length of the proximal tube 12 can be about 110 centimeters. A proximal tube 12 of this length may keep the proximal tube 12 in the straight portion of the guide. For use of catheter 10 as a guide wire, the outside diameter can be around 0.014 inches. For other applications requiring access to smaller bodily lumen, an outside diameter of less than 0.014 inches may be used.

The distal tube 16 is secured to the proximal tube 12. Surfaces of the distal tube 16 may be adhesively bonded or welded to surfaces of the proximal tube 12 where they contact or approach one another. The distal tube 16 generally guides a fluid from a first end 116 of distal tube 16 to a second end 116 of the distal tube 16 or to a location adjacent to the second end 216 of distal tube 16. The distal tube 16 may also be generally configured to permit the distal end 116 of guide tube 16 to be positioned at a desired location within a bodily lumen of a patient.

The distal tube 16 defines at least one distal lumen 26 to permit the communication of fluids along at least a portion of the length of the distal tube 16. The distal lumen 26 may also receive the core wire 14. When the distal lumen 26 is configured to receive a core wire 14, the distal lumen 26 is sized to accommodate the core wire 14 and to allow the communication of fluid along at least a portion of the length of the distal lumen 26. The distal lumen 26 may extend along at least a portion of the length of the distal tube 16. In one aspect, the distal lumen 26 of the distal tube 16 may extend longitudinally parallel to the longitudinal axis 300. In addition, the distal lumen 26 of the distal tube 26 may extend along the longitudinal axis 300. The distal lumen 26 may extend between a first distal tube opening 36 to a second distal tube opening 46 defined by the distal tube 16. The distal tube 16 may further define a distal outlet 56 in fluid communication with the distal lumen 26. The second end 212 of the proximal tube 12 is typically connected to the first end 116 of the distal tube 16 such that the proximal lumen 22 is in fluid communication with the distal lumen 26. The proximal lumen 22 and the distal lumen 26 together may form a continuous passage extending between a first end 112 of proximal tube 12 and a second end 216 of distal tube 16.

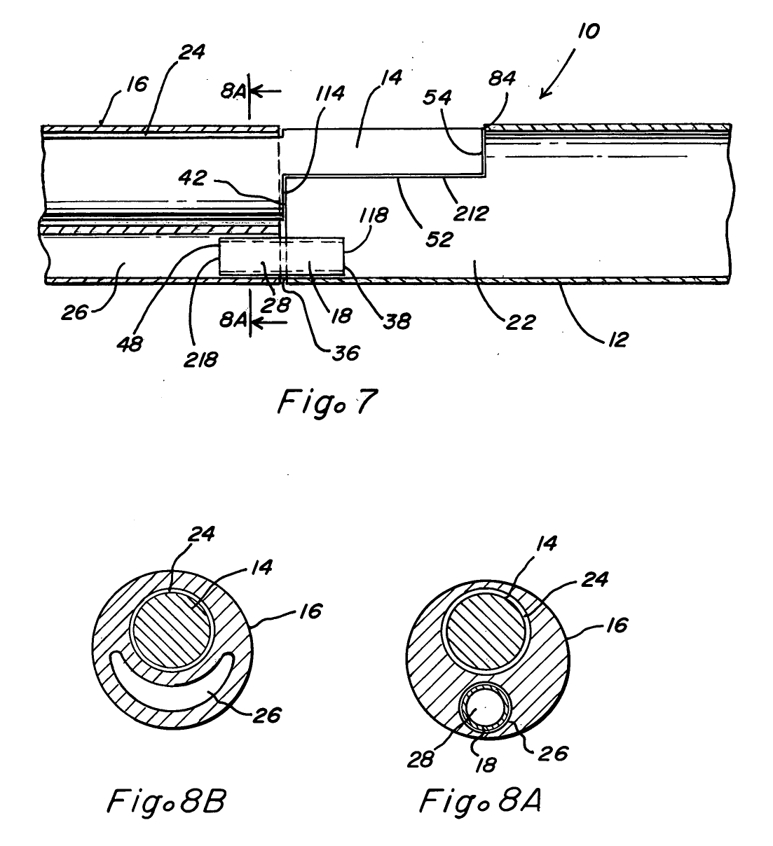

In addition, the distal tube 16 may define a core wire lumen 24 extending along at least a portion of the length of the distal tube 16 to receive the core wire 14. The core wire lumen 24 may extend between a first core wire opening 34 and a second core wire opening 44 defined by the distal tube 16. When a core wire lumen 24 is included in the distal tube 16, the core wire lumen 24 may be crescent shaped, as illustrated for exemplary purposes in FIG. 6B or otherwise shaped to optimize fluid flow characteristics.

The distal tube 16 may be made from a range of materials that will be recognized by those skilled in the art as depending upon the intended use for the resultant catheter 10. In one aspect, the distal tube 16 can be made from one or more polymers such as polyethylene, nylon, polyimide, among others. The distal tube may be solid as generally illustrated throughout the figures or may be a woven, braided material, or otherwise constructed as will be recognized by those skilled in the art upon review of the present disclosure.

The distal tube 16 typically has an outside diameter which is the same or smaller than the outside diameter of the proximal tube 12. The distal lumen 26 may be configured with as large a cross-sectional area as large as possible given the size and application for the catheter 10. For use primarily as a balloon catheter, the distal tube 16 may have an outside diameter of 0.024 inches. The distal lumen 26 may then have a diameter of about 0.020 inches. When the distal tube 16 includes a separate core wire lumen 24, the core wire lumen may have a diameter of 0.010 inches assuming the proximal core wire is 0.009.

A bridge tube 18 may extend between the proximal lumen 22 and the distal lumen 26. The bridge tube 18 defines a bridge lumen 28. The bridge lumen 28 typically communicates fluids between the proximal lumen 22 and the distal lumen 26. In one aspect, the bridge tube 18 may extend between the second proximal tube opening 42 and the first distal tube opening 46. The bridge lumen 28 may extend between first bridge tube opening 38 at a first end 118 of the bridge tube 18 and a second bridge tube opening 48 at a second end 218 of the bridge tube 18. The bridge tube 18 typically has a round external cross-sectional shape. However, the bridge tube 18 may have an external cross-sectional shape which corresponds to the shape of the lumen in which it may be received.

The bridge lumen 28 of the bridge tube 18 generally extends longitudinally within the bridge tube 18 from a first bridge tube opening 38 defined by the bridge tube 18 at a first end 118 of the bridge tube 18 to a second bridge tube opening 48 defined by the bridge tube 18 at a second end 218 of the bridge tube 18.

Typically, the bridge tube 18 is secured at a first end 118 in the proximal lumen 22 through the second proximal tube opening 42 of proximal tube 12 and at a second end 218 in the distal lumen 26 through the first distal tube opening 36 of distal tube 16. The bridge tube 18 may be secured within the proximal lumen 22 and distal lumen 26 by welding, adhesive bonding, or by mechanical interaction such as for example being compressionally fitted within proximal lumen 22 and distal lumen 26. When the bridge tube 18 does not sealingly engage the lumen in which it is positioned, such as for example when the external cross-sectional shape of the bridge tube 18 does not correspond to the shape of the proximal lumen 22 or the distal lumen 26, a sealing compound may be applied to seal any gaps. In one aspect, the adhesive compound used to adhesively bond the bridge tube 18 within the proximal lumen 22 and distal lumen 26 may also function as the sealing compound to seal any gaps between the walls of the proximal lumen 22 and distal lumen 26 and the outer surface of the bridge tube 18.

The core wire 14 extends from the second end 212 of proximal tube 12 and along at least a portion of the length of distal tube 16. The core wire 14 typically extends from a notch 52 in the proximal tube 12 through the core wire lumen 24 or the distal lumen 26 of the distal tube 16. In one aspect, the core wire 14 may function to modify the torquability and pushability of the distal tube 16. In other aspects, the core wire 14 may be used to, at least in part, secure the distal tube 16 to the proximal tube 12 of a catheter 10.

A first end 114 of the core wire 14 is secured to the notch 52 at the second end 212 of the proximal tube 12. In one aspect, the core wire 14 is received in a core wire lumen 24 or a distal lumen 26 of the distal tube 16. In this aspect, the core wire 14 may be secured within the core wire lumen 24 or the distal lumen 26. The core wire 14 may particularly be secured within one or more of the core wire lumen 24 and the distal lumen 26 at one or more locations proximal to the second end 216 of the distal tube or proximal to the balloon 20. The core wire 14 may be secured to the distal tube 16 solely at the second end 214 of the core wire 14 or at a location near the second end 214 of the core wire 14. In another aspect, the core wire 14 is received within a core wire lumen 24 or distal lumen 26 of the distal tube 16. The core wire may be rotatably and/or slidably received within the core wire lumen or distal lumen 26 of the distal tube 16. The core wire 14 may be secured within the lumen 24, 26 and may particularly be secured at a location proximal to the second end 216 of the distal tube 16 or proximal to the balloon 20. The core wire 14 may extend into or through the distal tube 16. The core wire 14 may extend to a location proximal the atraumatic tip 90. In one aspect, the core wire is secured at a second end 214 to the atraumatic tip 90. The core wire 14 generally assists in transmitting longitudinal motion and rotational movement from the proximal tube 12 to the second end 216 of the distal tube 16.

A core wire mating surface 54 is typically defined at the first end 114 of core wire 14 to be received by and secured within the notch 52 of the proximal tube 12. The core wire mating surface 54 may be formed along the length distal to the first end 114 of the core wire 14 which is modified so that the core wire 14 may have a cross-section which is only a portion of an arc of a circle. The core wire mating surface 54 may be sized and shaped to substantially match the flexibility of the region of the proximal tube 12 where the core wire mating surface 54 of core wire 14 overlaps and is secured within the notch 52 of the proximal tube 12. This matching of characteristics may reduce the likelihood of concentrated stresses resulting at kinks at the region where the core wire 14 is connected to the proximal tube 12.

As illustrated throughout the figures, core wire mating surface 54 is configured to extend into the notch 52 for a desired distance proximal to the second end 212 of the proximal tube 12. The core wire mating surface 54 may be sized and shaped to correspond to the notch 52 and may including one or more corresponding curved or angled surfaces and/or one or more planar surfaces. The core wire mating surfaces 54 may particularly include one or more longitudinal mating surfaces 74, perpendicular mating surfaces 84 and angled mating surfaces 94. In one aspect, the longitudinal mating surfaces 74, the perpendicular mating surfaces 84 and the angled mating surfaces 94 may each be planar. The angled mating surfaces 94 typically extend between five and ninety five degrees from the longitudinal axis 300. Generally, the longitudinal mating surfaces 74, perpendicular mating surfaces 84 and angled mating surfaces 94 will be shaped and proportioned to be secured to one or more of the respective longitudinal notch surface 72, perpendicular notch surface 82 and angled notch surface 94 of the core wire 14.

Typically, the core wire mating surface 54 is welded or adhesively bonded to the notch surfaces 62 of the proximal tube 12 defining the notch 52. This may provide torsional control and trackability similar to that of existing conventional guide wires and catheters which are well known and understood in the art for both function and design. Further, the core wire 14 may have regions of modified diameter, such as for example reduced, to change the flexibility of the core wire 14 at desired locations. In some aspects, this modification may improve the ability of the second end 216 of distal tube 16 to move through curves by minimizing friction between the distal tube 16 and the wall of the bodily lumen of the patent.

In addition, over a desired distance distal to the first end 114 of core wire 14 may also include one or more flow facilitating surfaces 64 as illustrated throughout the figures. The flow facilitating surfaces 64 may provide a flow path or enlarged flow path relative to provided about the adjacent circular portions of the core wire 14 through which fluid may travel from the proximal lumen 22 of the proximal tube 12 into the distal lumen 26 of the distal tube 16. In one aspect, the flow facilitating surfaces are coplanar to one or more of the core wire mating surfaces 54, such as for example the longitudinal mating surfaces 74 and the angled mating surfaces 94. In other aspects, the flow facilitating surface 64 may extend distally beyond the core wire mating surfaces to direct fluid distally within the distal lumen 26 around the periphery of the core wire 14.

The core wire 14 is typically a metal wire having a circular cross-section at one or more locations along the longitudinal axis of the core wire 14. The core wire 14 is typically made of a rigid but elastic material. Although the core wire 14 is typically made from a metal such as stainless steel or nitinol, the core wire 14 may be formed from other metals or polymers as will be recognized by those skilled in the art upon review of the present disclosure. The core wire 14 is typically a solid wire. However, the core wire 14 may be hollow along at least a portion of its length. The core wire 14 may also be in the form of a wound cable, a braided filament, or otherwise alternatively configured as will be recognized by those skilled in the art upon review of the present disclosure.

For intercranial applications, the core wire 14 may be about 40 centimeters long where the insertion point is in the femoral artery. In an exemplary embodiment where the proximal tube 12 has an outside diameter of 0.014 inches, the first end 114 of the core wire 14 can have a diameter of about 0.009 inches where it attaches to the proximal tube 12. The core wire 14 may include several reductions in diameter toward the second end 214 of core wire 14. In this aspect, the core wire 14 may have a diameter of about 0.004 inches at the second end 214 of the core wire 14.

A balloon 20 may be provided at an end or along the distal tube 16 for a variety of purposes including, vessel occlusion, stent expansion, and vessel dilation, among others. The balloon 20 will typically be positioned at or near the second end 216 of the distal tube 16. The balloon 20 may include an inflation chamber 30. In one exemplary aspect, the balloon 20 in the form of a membrane defining the inflation chamber 30 with a single opening and with the opening sealingly secured about the second end 216 of distal tube 16. In another exemplary aspect, the balloon 20 may be in the form of a tube sealingly attached at its ends to one or more of the distal tube 16 and the core wire 18. In one aspect, the second distal tube opening 46 may be in fluid communication with the inflation chamber 30 of the balloon 20 to permit a fluid such as for example inflation media to be introduced or removed from the inflation chamber 30. In another aspect, the distal outlet 56 may be in fluid communication with the inflation chamber 30 of the balloon 20 to permit a fluid such as for example inflation media to be introduced or removed from the inflation chamber 30. The inflation chamber 30 of the balloon 20 is typically in fluid communication with the second distal tube opening 46 to, for example, communicate inflation media to and/or from the inflation chamber 30. The inflation chamber 30 of the balloon 20 may alternatively or additionally be in fluid communication with the distal outlet 56 to again, for example, communicate inflation media to and/or from the inflation chamber 30.

Typically, the balloon will be made from a material such as silicone, latex, polyurethane, polyethylene, polypropylene or other material that will be recognized by those skilled in the art. The material may selected to have a high degree of biocompatibility. The balloon can be configured for to inflate to pressures between one (1) atmosphere and three (3) atmospheres. One atmosphere (1) is frequently used for the occlusion of blood vessels and three (3) atmospheres is frequently used for dilating vessels, expanding stents and the like. The balloon may also include a surface coating. In one aspect, the surface coating may make the balloon “grippier” under physiological conditions within a bodily lumen of a patient. Depending upon the application for the catheter 10, the balloon 20 may be either compliant or non-compliant. For vascular applications, the balloon form or tube of balloon 20 may be configured and sized to provide the correct inflated diameter and length for target vessel treatment locations. Target vessel diameters can range from as large as 10 to 12 millimeters to as small as 2 to 3 millimeters. Various lengths for the balloon 20 may be utilized as required for particular applications. In compliant embodiments, the balloon 20 may be made from silicone. For neurovascular applications, silicone may provide additional therapeutic benefits relating to spasms that will be recognized by those skilled in the art upon review of the present disclosure. When silicone is used, the silicone material may have a durometer of about 20 to 30. For neurovascular applications, this may give the catheter 10 the correct ‘feel’ when the balloon is inflated to a pressure of about 1 atmosphere.

An atraumatic tip 90 may be attached to the second end 114 of the core wire 14 or the second end 116 of the distal tube 16. The atraumatic tip 90 may be a spring coil. The spring coil may be about 2 cm long and about 0.014 inches in diameter. The spring coil may be made of 0.002 inches in diameter radio-opaque material, preferably platinum. However, other materials known in the art can be used as well. The atraumatic tip 90 generally provides a soft, gentle bumper for the second end 114 of the core wire 14 or the second end 116 of the distal tube 16. A shaping ribbon 98 may be positioned within the coil 96. The shaping ribbon 98 is typically constructed from a metal and can serve several important functions. The shaping ribbon 98 may serve as a bendable beam to more easily permit a user to induce a curved shape in the atraumatic tip 90 to direct the catheter 10 through a bodily lumen of a patient. When secured to core wire 14, the shaping ribbon 98 may transmit rotational motion from the second end 14 of the core wire 14 to the distal tip of the coil 96 of the atraumatic tip 90. Further, the shaping ribbon 98, may improve the safety of a catheter 10 by not allowing the coils 96 of the atraumatic tip 90 to stretch out if a portion of the atraumatic tip 90 becomes lodged or otherwise hung up in the bodily lumen of a patient. The first end of the shaping ribbon 98 may be attached to the second end 114 of the core wire 14 or the proximal ends of the coils 96 and the second end is secured to the distal end of the coils 96. The size of the shaping ribbon 98 may be about 0.002 inches by 0.004 inches. The shaping ribbon 98 is made from a material having the desired combination of ductility and elasticity. Stainless steel of a proper temper is commonly used to provide these characteristics. The spring coil 96 may terminate in a substantially hemispherical shaped cap so it is atraumatic to the wall of a bodily lumen. The cap secured to the distal end of the coils 96 and/or shaping ribbon 98, may be formed by melting the a distal portion of the coils, may be formed from a ball of solder or may be otherwise formed or secured as will be recognized by those skilled in the art upon review of the present disclosure.

An exemplary junction between a proximal tube 12 and a distal tube 16 in accordance with the present inventions is illustrated in FIG. 2 with aspects of similar embodiments illustrated in FIGS. 4 to 6C. As illustrated, the distal tube 16 includes only a distal lumen 26 for exemplary purposes. The distal lumen 26 receives the core wire 14 and carries the fluid along at least a portion of the length of distal tube 16. The illustrated embodiment further includes a bridge tube 18 extending between the proximal lumen 22 of proximal tube 12 and the distal lumen 26 of the distal tube 16. The bridge tube 18 is shown extending to a position proximal to the first end 114 of core wire 14. The second end 212 of the proximal tube 12 is configured to abut the first end 116 of the distal tube 16 when the core wire 14 is positioned within the distal lumen 26 and the core wire mating surface 54 is secured within the notch 52. The abutting ends may be welded together, adhesively bonded or otherwise secured to one another to seal the proximal lumen 22 and distal lumen 26 about the bridge tube 18. In addition or alternatively to the welded junctions, an adhesive compound and/or a sealing compound may be used to seal the proximal lumen 22 and distal lumen 26 about the bridge tube 18. The core wire mating surface 54 at the first end 114 of core wire 14 is secured to the notch surface 62 defining notch 52. As shown, core wire mating surface 54 includes a longitudinal mating surface 74 and a perpendicular mating surface 84 which are peripherally secured, as illustrated in FIG. 6B, within notch 52 of the proximal tube 12 at a longitudinal notch surface 72 and a perpendicular notch surface 82, respectively. The core wire mating surface 54 may be welded, adhesively bonded or otherwise secured to the notch surfaces 62 defining notch 52. As illustrated, the longitudinal mating surface 74 extends to the distal lumen 26 of distal tube 16 and transitions into a flow facilitating surface 64 which includes a curve to transition into the region of core wire 14 having a circular cross-section. The bridge tube 18 extends distally along the flattened surface 74 of the core wire mating surface 54 from the proximal lumen 22 of the proximal tube 12 into the distal lumen 26 of the distal tube 16. The second end 118 of the bridge tube 18 extends to a point approaching the curve. In other embodiments, the second end 118 of the bridge tube 18 may extend up to a point along the curve or may terminate at any point prior to the curve within the distal lumen 26 of the distal tube 16.

Another exemplary junction between a proximal tube 12 and a distal tube 16 in accordance with the present inventions is illustrated in FIG. 3. As illustrated, the distal tube 16 includes only a distal lumen 26. The distal lumen 26 receives the core wire 14 and carries the fluid along at least a portion of the length of distal tube 16. The illustrated embodiment again includes a bridge tube 18 extending between the proximal lumen 22 of proximal tube 12 and the distal lumen 26 of the distal tube 16. The bridge tube 18 is shown terminating at a position distal to the first end 114 of core wire 14. The second end 212 of the proximal tube 12 is configured to approach the first end 116 of the distal tube 16 when the core wire 14 is positioned within the distal lumen 26 and the core wire mating surface 54 is secured within the notch 52 defined by notch surfaces 62. The bridge tube 18 may be welded, adhesively bonded or otherwise secured within the second proximal tube opening 42 at a first end 118 of the bridge tube 18 and within the first distal tube opening 36 at a second end 218 of the bridge tube 18. In addition or alternatively, an adhesive compound and/or a sealing compound may be used to seal the proximal lumen 22 and distal lumen 26 about the bridge tube 18. The core wire mating surface 54 at the first end 114 of core wire 14 is secured to the notch 52. As shown, core wire mating surface 54 includes a longitudinal mating surface 74 and a perpendicular mating surface 84 which are peripherally secured to the corresponding notch surfaces 62 defining notch 52 in the proximal tube 12. The core wire mating surface 54 may be welded, adhesively bonded or otherwise secured to one or more notch surfaces 62 defining the notch 52. As illustrated, the flow facilitating surface 64 extends into the distal lumen 26 of distal tube 16 and transitions at abrupt angles into the region of core wire 14 having a circular cross-section. The bridge tube 18 extends distally along the flow facilitating surface 64 from the proximal lumen 22 of the proximal tube 12 into the distal lumen 26 of the distal tube 16. The second end 118 of the bridge tube 18 extends to a point approaching the angled transition. In other embodiments, the second end 118 10 of the bridge tube 18 may extend up to a point along the angled transition or may terminate at any point prior to the angled transition within the distal lumen 26 of the distal tube 16.

Another exemplary junction between a proximal tube 12 and a distal tube 16 in accordance with the present inventions is illustrated in FIG. 7. As illustrated, the distal tube 16 includes a distal lumen 26 and a core wire lumen 24. The distal lumen 26 is configured to communicate a fluid along at least a portion of the length of distal tube 16. FIGS. 8A and 8B illustrate varying configurations for the shape of the distal lumen 26. The distal lumen 26 configuration of FIG. 8B being a crescent shape to maximize the available flow cross-section. The core wire lumen 24 receives the core wire 14 along at least a portion of the length of the core wire lumen 24. The illustrated embodiment again includes a bridge tube 18 extending between the proximal lumen 22 of proximal tube 12 and the distal lumen 26 of the distal tube 16. The second end 212 of the proximal tube 12 is configured to again approach the first end 116 of the distal tube 16 when the core wire 14 is positioned within the distal lumen 26 and the core wire mating surface 54 is secured within the notch 52 to one or more notch surfaces 62. The bridge tube 18 may be welded, adhesively bonded, mechanically secured or otherwise secured within the second proximal tube opening 42 at a first end 118 of the bridge tube 18 and within the first distal tube opening 36 at a second end 218 of the bridge tube 18. In addition or alternatively, an adhesive compound and/or a sealing compound may be used to seal the proximal lumen 22 and distal lumen 26 about the bridge tube 18. The core wire mating surface 54 at the first end 114 of core wire 14 is secured to the notch 52. As shown, core wire mating surface 54 includes a longitudinal mating surface 74 and a perpendicular mating surface 84 which are peripherally secured, similar to those illustrated in FIG. 6B, to respective longitudinal notch surface 72 and perpendicular notch surface 82 defining notch 52 in the proximal tube 12. The core wire mating surface 54 may be welded, adhesively bonded or otherwise secured to the notch 52.

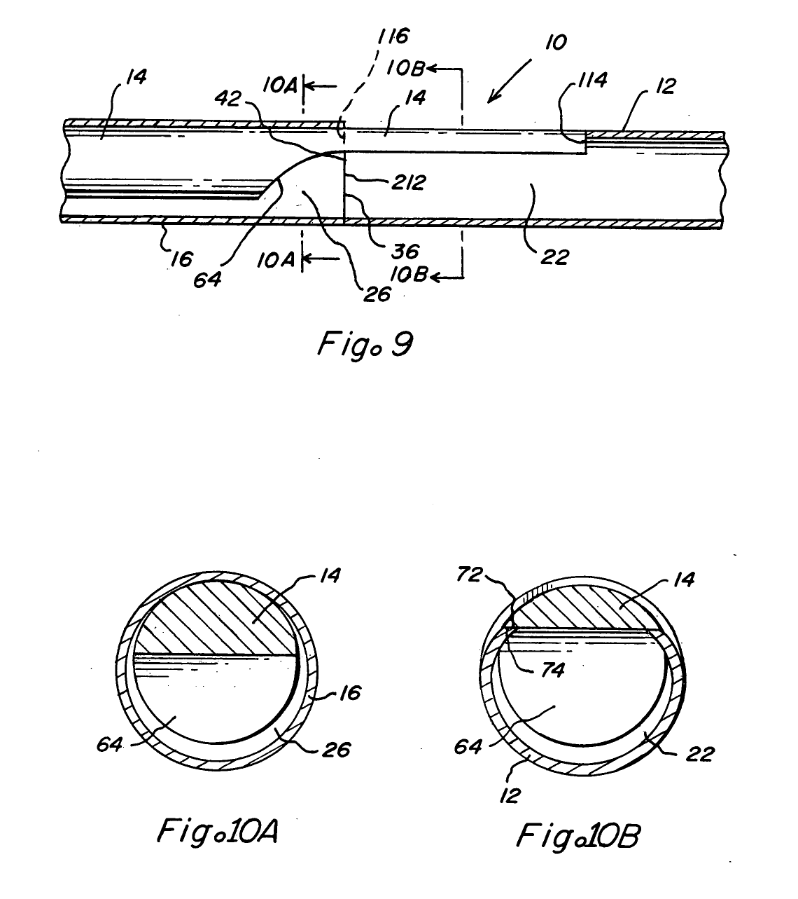

Another exemplary junction between a proximal tube 12 and a distal tube 16 in accordance with the present inventions is illustrated in FIG. 9. As illustrated, the distal tube 16 includes only a distal lumen 26. As illustrated, the distal lumen 26 receives the core wire 14 and carries the fluid along at least a portion of the length of distal tube 16. The illustrated embodiment does not include a bridge tube 18 extending between the proximal lumen 22 of proximal tube 12 and the distal lumen 26 of the distal tube 16. The second end 212 of the proximal tube 12 is configured to abut the first end 116 of the distal tube 16 when the core wire 14 is positioned within the distal lumen 26 and the core wire mating surface 54 is secured within the notch 52. The abutting ends may be welded together, adhesively bonded or otherwise secured to one another to seal the proximal lumen 22 and distal lumen 26 in fluid communication with one another. In addition or alternatively, welded junctions, an adhesive compound, and/or a sealing compound may be used to seal the proximal lumen 22 and distal lumen 26 in fluid communication with one another. The core wire mating surface 54 at the first end 114 of core wire 14 is secured to the notch surface 62 defining notch 52. As shown, core wire mating surface 54 includes a longitudinal mating surface 74 and a perpendicular mating surface 84 which are peripherally secured, as illustrated in FIGS. 9 and 10B, within notch 52 of the proximal tube 12 at a longitudinal notch surface 72 and a perpendicular notch surface 82, respectively. The core wire mating surface 54 may be welded, adhesively bonded or otherwise secured to the notch surfaces 62 defining notch 52. As illustrated, the longitudinal mating surface 74 extends to the distal lumen 26 of distal tube 16 and transitions into a flow facilitating surface 64 which includes a curve to transition into the region of core wire 14 having a circular cross-section. The core wire mating surface 54 may be welded, adhesively bonded or otherwise secured to the notch 52. As illustrated, the longitudinal flattened portion extends into the distal lumen 26 of distal tube 16 and transitions through a curve into the region of core wire 14 having a circular cross-section.

Yet another exemplary junction between a proximal tube 12 and a distal tube 16 in accordance with the present inventions is illustrated in FIG. 11. As illustrated, the distal tube 16 includes only a distal lumen 26. As illustrated, the distal lumen 26 receives the core wire 14 and carries the fluid along at least a portion of the length of distal tube 16. The particular illustrated embodiment again does not include a bridge tube 18 extending between the proximal lumen 22 of proximal tube 12 and the distal lumen 26 of the distal tube 16. The first end 116 of the distal tube 16 is configured to overlap the second end 212 of the proximal tube 12 when the core wire 14 is positioned within the distal lumen 26 and the core wire mating surface 54 is secured within the notch 52.

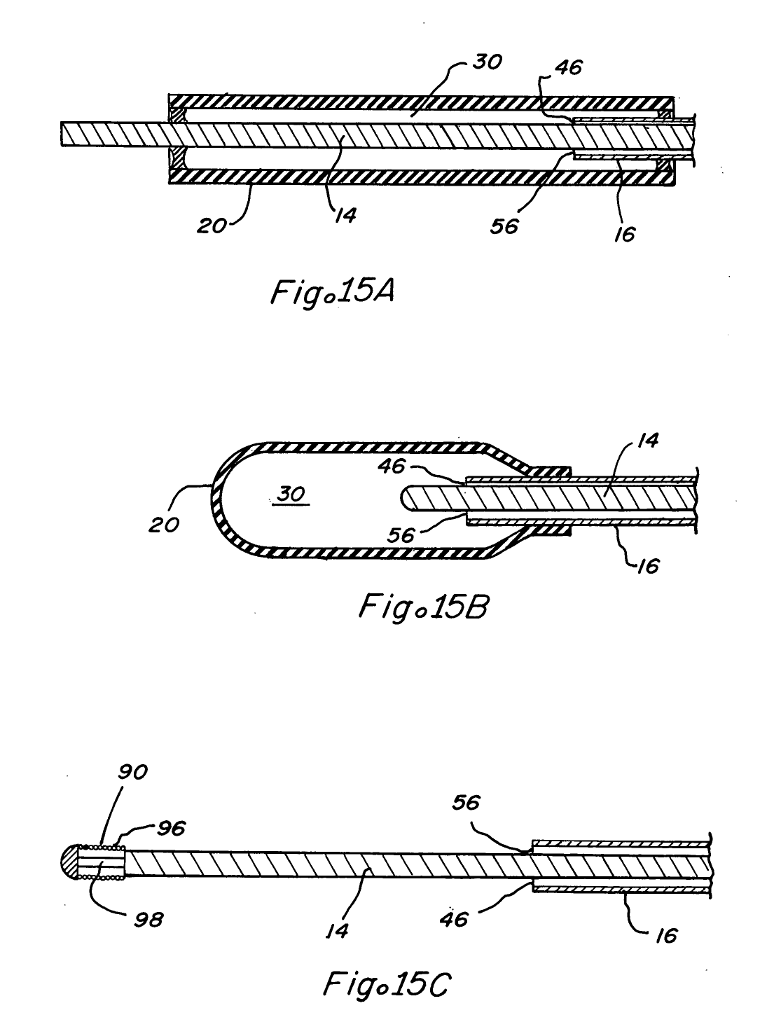

The core wire mating surface 54 at the first end 114 of core wire 14 is secured to the notch surface 62 defining notch 52. As shown, core wire mating surface 54 includes an angled mating surface 94 which are peripherally secured, as illustrated in FIGS. 11 and 14A, within notch 52 of the proximal tube 12 at an angled notch surface 92. The core wire mating surface 54 may be welded, adhesively bonded or otherwise secured to the notch surfaces 62 defining notch 52. As illustrated, the angled mating surface 94 extends to the distal lumen 26 of distal tube 16 and is coplanar with a flow facilitating surface 64. The core wire mating surface 54 may be welded, adhesively bonded or otherwise secured to the notch 52. As illustrated, the flow facilitating surface extends into the distal lumen 26 of distal tube 16 and transitions at an abrupt angle into the region of core wire 14 having a circular cross-section.

To use a catheter 10 in accordance with the present invention, a user may insert the distal end of catheter 10 into a body lumen of a patient using, for example, the Seldinger technique. The catheter 10 is guided through the body lumen to a location within the patient requiring treatment. As catheter 10 is guided through the patient, a user can manipulate the proximal tube 12 to direct the second end 216 of the distal tube 16 through the desired body lumen. When the second end 216 of the distal tube 16 is positioned at or near the location within the body lumen requiring treatment, the user may initiate the desired treatment. In embodiments where the catheter includes a balloon at or near the second end 216 of the distal tube 16, the balloon may be inflated to a desired size and/or pressure to affect the desired treatment. When the distal portion of the catheter 10 has an outside diameter of around 0.014 inches, small lumen such as various arteries and veins in the brain and heart may be accessed for diagnosis and/or treatment of the particular lumen or region. For example, in the illustrated embodiments, a catheter 10 including a balloon 20, properly sized and configured, may enable a user to access more distal or tortuous regions of the body.

Alternatively, after catheter 10 is guided to a desired location within the body, catheter 10 may be used to infuse fluid to that location. For example, fluids, such as saline solution, medicants, x-ray contrast liquids among other fluids, may be infused through second distal tube opening 46, and/or distal outlet 56.

Catheter 10 may further be used to guide surgical or diagnostic instruments over catheter 10 to access a desired location in a body lumen. When the instrument is positioned at the desired location within the body lumen, at least one surgical, therapeutic, or diagnostic procedure using the instrument is performed. The instrument may be removed and replaced with a different instrument as required by the treatment, diagnosis, or surgical procedure being performed by the user.

The foregoing discussion discloses and describes merely exemplary embodiments of the present invention. Upon review of the specification, one skilled in the art will readily recognize from such discussion, and from the accompanying drawings and claims, that various changes, modifications and variations can be made therein without departing from the spirit and scope of the invention as defined in the following claims.

What is claimed is:

1. An apparatus for accessing a bodily lumen of a patient, comprising:
a proximal tube defining a proximal lumen extending between a first end and a second end of the proximal tube, the second end of the proximal tube including a notch defined by a notch surface;
a core wire having a first end and a second end, the core wire including a core wire mating surface at the first end of the core wire, at least a portion of the core wire mating surface of the core wire secured to the notch surface of the proximal tube; and
a distal tube defining a distal lumen extending between a first end and a second end of the distal tube, the core wire received within the distal lumen of the distal tube and the distal lumen of the distal tube in fluid communication with the proximal lumen of the proximal tube.

2. An apparatus, as in claim 1, the notch surface defining the notch further comprising a longitudinal notch surface and the core wire mating surface further comprises a longitudinal mating surface, with the longitudinal notch surface secured to the longitudinal mating surface over at least a portion of the length of the longitudinal notch surface.

3. An apparatus, as in claim 1, the notch surface defining the notch further comprising a perpendicular notch surface and the core wire mating surface further comprises a perpendicular mating surface, with the perpendicular notch surface secured to the perpendicular mating surface over at least a portion of the length of the perpendicular notch surface.

4. An apparatus, as in claim 1, the notch surface defining the notch further comprising a longitudinal notch surface and a perpendicular notch surface and the core wire mating surface further comprises a longitudinal mating surface and a perpendicular mating surface, with the longitudinal notch surface secured to the longitudinal mating surface over at least a portion of the length of the longitudinal notch surface and with the perpendicular notch surface secured to the perpendicular mating surface over at least a portion of the length of the perpendicular notch surface.

5. An apparatus, as in claim 1, the notch surface defining the notch further comprising an angled notch surface and the core wire mating surface further comprises a angled mating surface, with the angled notch surface secured to the angled mating surface over at least a portion of the length of the angled notch surface.

6. An apparatus, as in claim 1, the core wire further comprising a flow facilitating surface.

7. An apparatus, as in claim 6, the notch surface defining the notch further comprising a longitudinal notch surface and the core wire mating surface further comprises a longitudinal mating surface, with the longitudinal notch surface secured to the longitudinal mating surface over at least a portion of the length of the longitudinal notch surface.

8. An apparatus, as in claim 6, the notch surface defining the notch further comprising a perpendicular notch surface and the core wire mating surface further comprises a perpendicular mating surface, with the perpendicular notch surface secured to the perpendicular mating surface over at least a portion of the length of the perpendicular notch surface.

9. An apparatus, as in claim 6, the notch surface defining the notch further comprising a longitudinal notch surface and a perpendicular notch surface and the core wire mating surface further comprises a longitudinal mating surface and a perpendicular mating surface, with the longitudinal notch surface secured to the longitudinal mating surface over at least a portion of the length of the longitudinal notch surface and with the perpendicular notch surface secured to the perpendicular mating surface over at least a portion of the length of the perpendicular notch surface.

10. An apparatus, as in claim 6, the notch surface defining the notch further comprising an angled notch surface and the core wire mating surface further comprises a angled mating surface, with the angled notch surface secured to the angled mating surface over at least a portion of the length of the angled notch surface.

11. An apparatus, as in claim 1, further comprising a bridge tube extending between the distal lumen of the distal tube and the proximal lumen of the proximal tube.

12. An apparatus, as in claim 11, the notch surface defining the notch further comprising a longitudinal notch surface and the core wire mating surface further comprises a longitudinal mating surface, with the longitudinal notch surface secured to the longitudinal mating surface over at least a portion of the length of the longitudinal notch surface.

13. An apparatus, as in claim 11, the notch surface defining the notch further comprising a perpendicular notch surface and the core wire mating surface further comprises a perpendicular mating surface, with the perpendicular notch surface secured to the perpendicular mating surface over at least a portion of the length of the perpendicular notch surface.

14. An apparatus, as in claim 11, the notch surface defining the notch further comprising a longitudinal notch surface and a perpendicular notch surface and the core wire mating surface further comprises a longitudinal mating surface and a perpendicular mating surface, with the longitudinal notch surface secured to the longitudinal mating surface over at least a portion of the length of the longitudinal notch surface and with the perpendicular notch surface secured to the perpendicular mating surface over at least a portion of the length of the perpendicular notch surface.

15. An apparatus, as in claim 11, the notch surface defining the notch further comprising an angled notch surface and the core wire mating surface further comprises a angled mating surface, with the angled notch surface secured to the angled mating surface over at least a portion of the length of the angled notch surface.

16. An apparatus for accessing a bodily lumen of a patient, comprising:
a proximal tube defining a proximal lumen extending between a first end and a second end of the proximal tube, the second end of the proximal tube including a notch;
a core wire having a first end and a second end, the core wire including a core wire mating surface extending to the first end of the core wire, the core wire mating surface of the core wire secured to the notch in the proximal tube; and
a distal tube defining a distal lumen and a core wire lumen, the core wire received within the core wire lumen of the distal tube and the distal lumen of the distal tube in fluid communication with the proximal lumen of the proximal tube.

17. An apparatus, as in claim 16, the notch surface defining the notch further comprising a longitudinal notch surface and the core wire mating surface further comprises a longitudinal mating surface, with the longitudinal notch surface secured to the longitudinal mating surface over at least a portion of the length of the longitudinal notch surface.

18. An apparatus, as in claim 16, the notch surface defining the notch further comprising a perpendicular notch surface and the core wire mating surface further comprises a perpendicular mating surface, with the perpendicular notch surface secured to the perpendicular mating surface over at least a portion of the length of the perpendicular notch surface.

19. An apparatus, as in claim 16, the notch surface defining the notch further comprising a longitudinal notch surface and a perpendicular notch surface and the core wire mating surface further comprises a longitudinal mating surface and a perpendicular mating surface, with the longitudinal notch surface secured to the longitudinal mating surface over at least a portion of the length of the longitudinal notch surface and with the perpendicular notch surface secured to the perpendicular mating surface over at least a portion of the length of the perpendicular notch surface.

20. An apparatus, as in claim 16, the notch surface defining the notch further comprising an angled notch surface and the core wire mating surface further comprises a angled mating surface, with the angled notch surface secured to the angled mating surface over at least a portion of the length of the angled notch surface.

21. An apparatus, as in claim 16, the core wire further comprising a flow facilitating surface.

22. An apparatus, as in claim 21, the notch surface defining the notch further comprising a longitudinal notch surface and the core wire mating surface further comprises a longitudinal mating surface, with the longitudinal notch surface secured to the longitudinal mating surface over at least a portion of the length of the longitudinal notch surface.

23. An apparatus, as in claim 21, the notch surface defining the notch further comprising a perpendicular notch surface and the core wire mating surface further comprises a perpendicular mating surface, with the perpendicular notch surface secured to the perpendicular mating surface over at least a portion of the length of the perpendicular notch surface.

24. An apparatus, as in claim 21, the notch surface defining the notch further comprising a longitudinal notch surface and a perpendicular notch surface and the core wire mating surface further comprises a longitudinal mating surface and a perpendicular mating surface, with the longitudinal notch surface secured to the longitudinal mating surface over at least a portion of the length of the longitudinal notch surface and with the perpendicular notch surface secured to the perpendicular mating surface over at least a portion of the length of the perpendicular notch surface.

25. An apparatus, as in claim 21, the notch surface defining the notch further comprising an angled notch surface and the core wire mating surface further comprises a angled mating surface, with the angled notch surface secured to the angled mating surface over at least a portion of the length of the angled notch surface.

26. An apparatus, as in claim 16, further comprising a bridge tube extending between the distal lumen of the distal tube and the proximal lumen of the proximal tube.

27. An apparatus, as in claim 26, the notch surface defining the notch further comprising a longitudinal notch surface and the core wire mating surface further comprises a longitudinal mating surface, with the longitudinal notch surface secured to the longitudinal mating surface over at least a portion of the length of the longitudinal notch surface.

28. An apparatus, as in claim 26, the notch surface defining the notch further comprising a perpendicular notch surface and the core wire mating surface further comprises a perpendicular mating surface, with the perpendicular notch surface secured to the perpendicular mating surface over at least a portion of the length of the perpendicular notch surface.

29. An apparatus, as in claim 26, the notch surface defining the notch further comprising a longitudinal notch surface and a perpendicular notch surface and the core wire mating surface further comprises a longitudinal mating surface and a perpendicular mating surface, with the longitudinal notch surface secured to the longitudinal mating surface over at least a portion of the length of the longitudinal notch surface and with the perpendicular notch surface secured to the perpendicular mating surface over at least a portion of the length of the perpendicular notch surface.

30. An apparatus, as in claim 26, the notch surface defining the notch further comprising an angled notch surface and the core wire mating surface further comprises a angled mating surface, with the angled notch surface secured to the angled mating surface over at least a portion of the length of the angled notch surface.
